(19) United States
(12) Patent Application Publication
(10) Pub. No.: US 2014/0235487 A1
(43) Pub. Date: Aug. 21, 2014

(54) ORAL CANCER RISK SCORING

(71) Applicant: William Marsh Rice University, Houston, TX (US)

(72) Inventors: John T. McDevitt, Houston, TX (US); Pierre N. FLORIANO, Houston, TX (US); Tim ABRAM, Houston, TX (US)

(73) Assignee: William Marsh Rice University, Houston, TX (US)

(21) Appl. No.: 14/261,670

(22) Filed: Apr. 25, 2014

Related U.S. Application Data
(63) Continuation-in-part of application No. PCT/US2011/060453, filed on Nov. 11, 2011.
(60) Provisional application No. 61/816,083, filed on Apr. 25, 2013.
(60) Provisional application No. 61/413,107, filed on Nov. 12, 2010.

(51) Int. Cl. G06F 19/24; G06F 19/00; G01N 33/574

(52) U.S. Cl. CPC G06F 19/24; G01N 33/57407; G06F 19/3418
USPC 506/9; 702/19

(57) ABSTRACT

Neural net method of computing oral cancer risk based on inputs such as age, gender, smoking status, morphological characteristics of sampled cells, and levels of biomarkers in samples cells.

### PRIOR RELATED APPLICATIONS

This application claims priority to U.S. Ser. No. 61/413,107, filed Nov. 12, 2010 and PCT/US2011/060453, filed Nov. 11, 2011, and also to U.S. Ser. No. 61/816,083, filed Apr. 25, 2013. Each of these applications is incorporated by reference in its entirety for all purposes.

### FEDERALLY SPONSORED RESEARCH STATEMENT

This invention was made with government support under Grant No. RC2-DE020785, awarded by the NIH. The government has certain rights in the invention.

### FIELD OF THE DISCLOSURE

This disclosure generally relates to methods, devices, disposables and systems for point of care diagnosis of oral cancer. In particular, methods of computing a risk score are provided, which includes demographic, morphogenic and biomarker input.

### BACKGROUND OF THE DISCLOSURE

All squamous cell carcinoma lesions are thought to begin via the repeated, uncontrolled division of cancer stem cells of epithelial lineage or characteristics. Accumulation of these cancer cells cause a microscopic focus of abnormal cells that are, at least initially, locally confined within the specific tissue in which the progenitor cell resided. This condition is called squamous cell carcinoma in situ, and it is diagnosed when the tumor has not yet penetrated the basement membrane or other delimiting structure to invade adjacent tissues. Once the lesion has grown and progressed to the point where it has breached, penetrated, and infiltrated adjacent structures, it is referred to as “invasive” squamous cell carcinoma. Once a carcinoma becomes invasive, it is able to spread to other organs and cause a metastasis or secondary tumor to form.

Oral cancer is a subtype of head and neck cancer and is any cancerous tissue growth located in the oral cavity. It may arise as a primary lesion originating in any of the oral tissues, by metastasis from a distant site of origin, or by extension from a neighboring anatomic structure, such as the nasal cavity. Oral cancers may originate in any of the tissues of the mouth, and may be of varied histologic types: teratoma, adenocarcinoma derived from a major or minor salivary gland, lymphoma from tonsillar or other lymphoid tissue, or melanoma from the pigment-producing cells of the oral mucosa. There are several types of oral cancers, but around 90% of diagnoses cases are squamous cell carcinomas, originating in the tissues that line the mouth and lips.

Oral squamous cell carcinoma (OSCC) is a global health problem afflicting close to 300,000 people each year. Despite significant advances in surgical procedures and treatment, the long-term prognosis for patients with OSCC remains poor, with a 5-year survival rate at approximately 50%, which is among the lowest for all major cancers. High mortality associated with OSCC is often attributed to advanced disease stage at diagnosis, underscoring the need for new diagnostic methods targeting early tumor progression and malignant transformations.

### SUMMARY OF THE DISCLOSURE

This disclosure describes an improvement upon previously disclosed “Detecting Tumor Biomarker in Oral Cancer” in WO2012065117 (App. No. PCT/US2011/060453). Herein, a score is created that integrates multiple measurements from demographic, morphological indicators, and biomarkers and provides a graded scale of disease conditions, ranging from benign to malignant. Importantly, the scoring is based on single cell input data, rather than an average signal produced by collections of cells, wherein important cancer signals can be masked by a preponderance of healthy cells.

Our previous disclosure taught the art of discriminating between two binary categories, one categorized as case, the other as non-case, through logistic regression. However, such results are limited in the information provided, and a much higher level of discrimination would be beneficial to clinicians and patients. Further, because individual cells are assayed, the discriminatory power of our data is much higher than could ever be realized before, making is possible to realize a graded scale of disease progression.

This new disclosure integrates the results (morphological, biomarker and demographics information) from multiple binary classifications as inputs, according to 3-way ordinal and 5-way ordinal scales of disease progression to create a continuous numerical scale, which will guide clinicians in their management of patients with potentially malignant lesions.

There are multiple machine learning techniques that can be employed herein, but artificial neural networks or logistic regression methods are preferred, e.g., a 2, 3, 4, 5 or more parameter logistic regression. The ultimate laboratory user can continue to use machine based learning techniques to include e.g., more data with time, thus refining the mathematical calculations, or to add in new data points, such as newly discovered biomarkers. However, this is not essential, and the user can instead simply employ final weighted values for each marker.

In preferred embodiments, a suspension of cells is collected with a rotating brush. See e.g., FIG. 3. Our research has indicated that collecting cells in this way is sufficient to permeabilize the cells for our purposes, but it is also possible to fix and permeabilize the whole cells in the usual way. The cells can be collected on a membrane that allows debris to pass through, but not whole cells. Alternatively, it is also possible to enrich for a particular population of cells with e.g., magnetic beads coupled, e.g., to a receptor or cell surface proteins, such as an antibody for EGFR. Then when the magnet is turned off, the enriched cancer cells can pass to the image cytometer. Using this technique, we have already been able to detect a single cell in 10,000 and we expect that we can easily get to 10⁶ and 10⁹ sensitivity when fully optimized.

The system then detects a variety of morphological and biological markers in individual cells, including for example, DAPI for DNA, and phalloidin for F-actin. These two stains provide a great deal of information about cell morphology, and for example, nuclear to cytoplasm ratio (an important indicator that a cell is transforming) and cell shape (cancer cells are rounder). Other parameters that can be measured and used in the model include but are not limited to:

Area (WCArea[red]): Area of Whole cell selection in square pixels determined in red from Phalloidin stain.

Mean Intensity Value (WCMean[red], [green]): Average value within the WC selection. This is the sum of the intensity values of all the pixels in the selection divided by the number of pixels. [red] has QA/QC value and [blue] has limited descriptive value, whereas [green] is the most important for surface markers. For intracellular markers, the NuMean[green] is most descriptive.

Standard Deviation (WCStdDev[red], [green]): Standard deviation of the intensity values used to generate the mean intensity value. [red] useful for Phalloidin, QA/QC and descriptive, [green] for surface markers.

Modal Value (WCMode[red], [green]): Most frequently occurring value within the selection. Corresponds to the highest peak in the histogram. Similar to Mean in terms of value.

Min & Max Level (WCMin and WCMax[red], [green], [blue]): Minimum and maximum intensity values within the selection. Limited descriptive value, may be used for QA/QC.

Integrated Density (WCIntDen[red], [green], [blue]): Calculates and displays “IntDen” (the product of Area and Mean Gray Value)−Dependent values.

Median (WCMedian[red], [green]): The median value of the pixels in the image or selection. This again is similar to Mean and Mode in terms of utility.

Circ. (circularity): 4π*area/perimeter²: A value of 1.0 indicates a perfect circle. As the value approaches 0.0, it indicates an increasingly elongated shape. Values may not be valid for very small particles.

AR (aspect ratio): diameters of major_axis/minor_axis.

Round (roundness): 4*area/(π*major_axis²): Could also use the inverse of the aspect ratio.

Other parameters may include percentages of cells with one or more parameters meeting certain criteria, or above a certain cut-off. Thus, a patient with 10% MCM2 cells may be better off than a patient with 32% MCM2 cells, or a rapid progression of MCM2-containing cells between sampling may indicate rapid disease progression, and the like. With prior multicellular-based assays, such detail in these few cells would be masked by the data of the rest of the sample.

Cells can also be stained with labeled antibodies for the various cancer markers discussed herein. Generally, different biomarkers should be labeled with different labels, so that they can be distinguished. However, some overlap is allowable where the markers are spatially distinguished in the cell, e.g., EGFR on the cell surface and Ki67 in the nucleus. Alternatively, the chip can be divided into two or three portions (or two chips used) and separate groups of labels employed.

As yet another alternative, the initial analysis can be on a whole cell basis, then the cells lysed and studied, and this may provide additional information about intracellular antigens. Of course, the data would then be an average over the cells in the sample, unless the cells are fixed in a particular location and the cell contents do not mix.

This disclosure also describes an expanded panel of biomarkers to cover early detection and progression of oral cancer. We analyze cellular samples obtained from a minimally invasive brush biopsy sample, simultaneously quantifying cell morphometric data and expression of molecular biomarkers including AVB6, EGFR, Ki67, Geminin, CD147, MCM2, Beta Catenin, and EMPPRIN.

The following abbreviations are used herein:

Abbreviations

| | |
|---|---|
| Ab | Antibody |
| ABS | Acrylonitrile butadiene styrene |
| AUC | Area under the curve |
| AVB6 or αVβ6 | Alpha V beta 6, an integrin |
| Beta-catenin | β Catenin is a protein that in humans is encoded by the |
| | CTNNB1 gene |
| BM | Biomarker |
| DNA | Deoxyribonucleic acid |
| EGFR | Epidermal Growth Factor Receptor |
| EMMPRIN | Extracellular Matrix Metalloproteinase Inducer, |
| | aka CD147 |
| FITC | Fluorescein isothiocyanate |
| HNSCC | Head and neck squamous cell carcinomas |
| IVD | in vitro diagnostic device |
| Ki67 | Antigen KI-67 also known as Ki-67 or MKI67 is a |
| | protein that in humans is encoded by the MKI67 gene |
| LOD | Limits of detection |
| MAB | Monoclonal Ab |
| MCM2 | Mini Chromosome Maintenance protein 2 |
| N/C ratio | nuclear/cytoplasmic ratio |
| BNC | Bionanochip |
| NPV or PV⁻ | Negative predictive value |
| NRAT | Nuclear to Cytoplasm ratio |
| NSE | Neuron-specific enolase |
| NUAR | Nuclear Area |
| OSCC | oral squamous cell carcinoma |
| PBSA | Phosphate buffered saline with bovine serum albumin |
| PML | Potentially malignant lesions |
| PBNC | Programmable BNC |
| PPV or PV⁺ | positive predictive value |
| PV | Predictive value |
| RNA | Ribonucleic acid |
| ROC | Receiver operating characteristic. A graphical plot |
| | of the sensitivity, or true positive rate, vs. false |
| | positive rate (1—specificity or 1—true negative |
| | rate), for a binary classifier system as its discrim- |
| | ination threshold is varied. The ROC can also be |
| | represented equivalently by plotting the fraction of |
| | true positives out of the positives (TPR = true positive |
| | rate) vs. the fraction of false positives out of the |
| | negatives (FPR = false positive rate). Also known as a |
| | Relative Operating Characteristic curve, because |
| | it is a comparison of two operating characteristics |
| | (TPR & FPR) as the criterion changes. |
| ROI | Region of interest |
| SUSP | A filter that flags suspicious cell according to |
| | morphometric and molecular parameters |
| WBCi | A filter that flags infiltrated white blood cells |
| | according to morphometric and molecular parameters |
| WCAR | Whole cell area, i.e. cytoplasm area |
| WCIR | Whole cell circularity |
| Wnt | Proto-oncogene protein Wnt |

The disclosure includes one or more the following embodiments, in any combination thereof:
A method of scoring oral cancer lesions comprising inputting the following data points into a computer:

one or more morphological characteristics from individual oral cells from a patient, said morphological characteristics selected from nuclear area, cell area, cell circularity, cell aspect ratio, and cell roundness;

one or more of gender, age, alcohol intake, and smoking status of said patient;

one or more biomarker levels from individual oral cells from said patient, said biomarker selected from the group consisting of alpha V beta 6 (AVB6), Epidermal Growth Factor Receptor (EGFR), Ki67, Geminin, Mini Chromosome Maintenance protein (MCM2), beta catenin, EMPPRIN, CD147;

calculating a risk score based on each of the above inputs, said risk score allowing a user to distinguish at least the following: i) benign lesions, ii) dysplastic lesions, and iii) cancerous lesions; and

displaying said risk score on an output device.
A method of scoring oral cancer lesions comprising inputting the following data points into a computer:

two, three or more morphological characteristics from individual oral cells from a patient, said morphological characteristics selected from cell area, nuclear area, cell circularity, cell aspect ratio, and cell roundness;

two, three or more of gender, age, alcohol intake, and smoking status of said patient;

two, three or more biomarker levels from individual oral cells from said patient, said biomarker selected from the group consisting of AVB6, EGFR, Ki67, MCM2, beta catenin, EMPPRIN, and CD147; and

calculating a risk score based on each of the above inputs, wherein said calculation is based on logistic regression or neural network training using data points from patients with known disease status, said risk score providing at least 3 disease classifications; and

displaying said risk score on an output device.
A method of detecting oral cancer and scoring oral lesions comprising:

obtaining an oral sample from a patient suspected of having an oral lesion, said oral sample containing a plurality of cells;

determining a cell area, a nuclear area, and a level of AVB6, MCM2, Ki67 and CD147 in each of said plurality of cells;

inputting the following data points into a computer:

said determined cell area and said determined nuclear area for each of said plurality of cells;

three or more of gender, age, alcohol intake, and smoking status of said patient;

said determined AVB6, MCM2, Ki67 and CD147 levels for each of said plurality of cells; and

calculating a risk score based on each of the above data points; and

displaying said risk score on an output device, wherein said risk score distinguishes at least three disease states.
A method wherein said calculation results in 4-way, 5-way or 6 way ordinal scales of disease progression.
A method wherein said calculation allows a user to distinguish the following: 1) benign lesions, 2) mild dysplasia, 3) moderate dysplasia, 4) severe dysplasia, and 5) oral squamous cell carcinoma (OSCC) or to distinguish the following: 1) benign lesions, 2) mild dysplasia, 3) moderate dysplasia, 4) severe dysplasia, and 5) oral squamous cell carcinoma (OSCC) combined with carcinoma in situ (CIS).
A method allowing a user to distinguish between benign conditions, mild dysplastic conditions, moderate dysplastic conditions, severe dysplastic conditions and cancerous conditions or allowing a user to distinguish the following: 1) benign conditions, 2) dysplastic conditions, 3) moderate disease, 4) high risk disease.
A method wherein said calculation is based on artificial neural nets, logistic regression, linear discriminate analysis, or random forests or based on feed forward artificial neural nets;
A method wherein the calculation is based on prior artificial neural network model training using data points from patients with known disease states or is based on continued neural network model training using data points from patients with known disease states and outcomes.
A method wherein each inputted data point corresponds to a node, and each node is linked to serve as input in a neural network in creating a single output risk score on a continuous scale between 1 and 10.
A method wherein said calculation is based on inputting nodes into an input layer, said nodes obtained through logistic regression of all possible classifications of patient samples having known disease states according to at least 3-way classifications; optimizing the artificial neural network as to the number of hidden layers and computing nodes, and outputting a normalized score between 1 and 10, 1 corresponding to benign and 10 corresponding to malignant.
A method, said calculation including: Oral Cancer Risk Score=a0+a1×P1+a2×P2+ . . . an X Pn, where each of P1, P2, . . . Pn is a node of a logistic regression model, where n is the number of nodes and where a0-an is a weight factor determined by training with input data from patients having known disease status.
A method, said calculation including levels of AVB6 and MCM2 or including cell area, nuclear area, and levels of AVB6, MCM2, Ki67 and CD147.

The word “morphometric” as used herein means the measurement of such cellular shape or morphological characteristics as cell shape, size, nuclear to cytoplasm ratio, membrane to volume ratio, and the like.

The phrase “based on” includes both contemporaneous use as well as prior use to establish parameter weights. Thus, a calculation based on earlier data training using neural nets would still be “based on” such neural net analysis, even if this part of the computational analysis does not need to be repeated.

The phrase “each of said plurality of cells” is meant to refer to individually testing each of the cells in at least a portion of a sample that is inputted into a measuring device, but excluding cell loss due to lysis and any losses to due excess sample not being tested. By individual testing, what is meant is that data is collected that is unique to each cell, nevertheless many cell images can be captured in a single photograph.

Nuclear to cytoplasmic ratio is calculated based on cell area and nuclear area e.g., NA/CA-NA.

The word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed, and excludes all additional elements.

The phrase “consisting essentially of” excludes additional material elements, but allows the inclusions of non-material elements that do not substantially change the nature of the disclosed methods.

### DESCRIPTION OF FIGURES

FIG. 1 Schematic representation of disease scale, herein normalized from 1-10, with 10 being disease and 1 being benign. The various “nodes’ are also illustrated.

FIG. 2. Schematic showing artificial neural network architecture, with various morphometric and biomarker inputs on the left and an OSCC score output on the right.

FIG. 3. Shows the overall process from sample collection, processing, analysis by lab-on-chip, to image processing, and data analysis.

FIG. 4A shows a prototype lab setup used for initial testing, and 4B shows a embodiment being developed for commercial sale. The commercial embodiment will include an analyzer that contains pumping systems for fluidics, as well as microscope and camera or CCD, light source, etc., and is used with disposable assay cartridges that allow analysis of single cells.

FIG. 5 shows image analysis, described more completely in WO2012065117, but which basically shows a conceptual flowchart of the image collection and conditioning process before measurements are performed on individual cell profiles.

FIG. 6 shows our single cell processing capability, beginning with taking 25 pictures per assay, individually locating each cell, and then analyzing the various parameters for each cell. As of the date of this figure, there were only 6 million cells collected and 500 processed, but now we have processed the data for over 10 million cells.

FIG. 7 shows a visual depiction of the back-end data processing components of the neural net architecture.

FIG. 8 shows the next generation system wherein images are collected at one site, analyzed in the cloud, and the final result sent to the pathologist or physician.

FIG. 9 shows a radar chart that illustrates the contributions of various summary statistics (mean, median, standard deviation, variance, etc.) of the cellular populations for different biomarkers corresponding to three different model classes: benign vs malignant (BvM), dysplastic vs malignant (DvM), and benign vs dysplastic (BvD).

FIG. 10 shows a similar radar chart as seen in FIG. 9, but illustrates morphological parameters corresponding to the same three model classes.

FIG. 11 shows the relative AUC values for the 5 best 2-parameter models for 3 model classes: BvD, BvM, and DvM. Additionally, a bar graph is included to emphasize the best AUC value.

FIG. 12 shows the relative AUC values for the 5 best 2-parameter models for 3 model classes: x23, x34, and x45. Additionally, a bar graph is included to emphasize the best AUC value.

FIG. 13 shows a bar graph that displays the AUC's for ROC curves derived from 6 different 3-parameter models.

### DETAILED DESCRIPTION

The following detailed description serves to illustrate various embodiments of the disclosure, but is not be used to unduly limit the claims and their equivalents.

Typically, in “classification” models, a single measure is collected per biomarker in each sample (e.g. panel of molecular biomarkers concentrations, or morphologic biomarker measures). The current study is atypical in that the biomarkers are measured for each cell, resulting in hundreds to thousands of measures per biomarker per sample. Thus, each biomarker has an entire distribution of measures per sample.

These distributions of biomarker values are further complicated by the fact that the cells within a sample may be heterogeneous, with some cells being benign and other cells being dysplastic or malignant. A homogeneous sample cells would likely have a bell-shaped distribution on either the arithmetic or logarithmic scales. However, a sample with a heterogeneous mixture of cells types would likely (if the biomarker had good discriminatory properties) be skewed or bi-modal in distribution.

For example, suppose a specific biomarker concentration increased substantially with malignancy and the cells of the sample were 27% malignant and 73% dysplastic. Then, this biomarker's median concentration (the 50th percentile) would encompass the biomarker concentration of dysplasia and completely miss the malignancy. Likewise, the effects of the 27% malignant cells on the mean biomarker concentration would be diluted by the 73% of the cells with dysplasia.

However, the 75th percentile of this biomarker's concentration should not be influenced by the dysplastic cell in the sample and be malignant in profile. Likewise, the heterogeneous mixture of cell types may increase the biomarker's variance, standard deviation, coefficient of variability (cv), interquartile range, flatness (kurtosis), and skewness.

Thus, given the unique nature of our cell-specific data, in summarizing biomarker concentration over all cells within a sample, it is useful to try multiple measures of the biomarker distribution in fitting the statistical models. Each biomarker was summarized using the following distributional measures:

| | |
|---|---|
| 1. | Mean |
| 2. | Median |
| 3. | Variance |
| 4. | Standard deviation |
| 5. | Coefficient of variation (cv) |
| 6. | Skewness |
| 7. | Kurtosis (any measure of the “peakedness” |
| | of the probability distribution) |
| 8. | 10th Percentile |
| 9. | 25th Percentile |
| 10. | 75th Percentile |
| 11. | 90th Percentile |
| 12. | >0.5 Z-Score (percent of cells with biomarker values greater |
| | than 0.5 standard deviations away from healthy cells) |
| 13. | >2.0 Z-Score (percent of cells with biomarker values greater |
| | than 2.0 standard deviations away from healthy cells) |
| 14. | >3.0 Z-Score (percent of cells with biomarker values greater |
| | than 3.0 standard deviations away from healthy cells) |

A 1000-patient characterization/association trial was run and recruitment completed with patients who presented with potentially malignant lesions. These lesions were brushed and analyzed with the methodology previously disclosed in WO2012065117, and also biopsied with a scalpel, so histopathology of the lesions could be conducted on slides by expert oral pathologists.

Diagnoses were established from the review of two pathologists on the same set of slides, and when they disagreed, a third pathologist served as the adjudicator to classify the lesions into one of 6 classes according the WHO guidelines. These categories included controls (1), benign (2), mild dysplasia (3), moderate dysplasia (4), severe dysplasia (5), and oral squamous cell carcinoma (OSCC) combined with carcinoma in situ, i.e. CIS. (6). Because CIS are rarer, we did not recruit a statistically significant number of these patients, and because as part of standard of care they are treated as the malignant lesions, they were bundled with OSCC. However, as our data set continues to increase (now at about 10 millions cells assayed), these will be separable into separate disease states.

Biomarker measurements including but not limited to intensity, or biomarker index (% of positive cells per patient/assay based on comparison of each cell's intensity to the intensity of the Control population for that particular biomarker), as well as morphological measurements, including but not limited to nuclear area, cell area, nuclear to cytoplasm ratio distribution, indices, or mean, are combined to establish the largest area under the curve (AUC), or ability to discriminate between two classes, one defined as the cases, the other as the non-cases. As such, we can obtain through combination of various morphological markers as well as molecular biomarkers, demographic and behavioral data, a logit score, product of the logistic regression equation using a weighed sum of all selected parameters. However, in our previous approach, this only allowed us to determine whether a particular patient belongs to one group or another, based for example on cases being OSCC and non-cases being benign.

This disclosure, by contrast, consists of the linkage of all possible created logit scores, that will be referred to as nodes, to serve as input in a mathematical algorithm, or artificial neural network in creating a single output OSCC risk score on a continuous scale between 1 and 10.

The term “neural network” was traditionally used to refer to a network or circuit of biological neurons, however, modern usage of the term often refers to artificial neural networks, which are composed of artificial neurons or nodes. Thus, the term as used herein refers to artificial neural networks for solving artificial intelligence problems.

An artificial neural network (ANN), often just called a neural network (NN), consists of an interconnected group of artificial neurons, and it processes information using a connectionist approach to computation. In most cases a neural network is an adaptive system changing its structure during a learning phase. Neural networks are used for modeling complex relationships between inputs and outputs or to find patterns in data. Neural Networks have several unique advantages as tools for cancer prediction. A very important feature of these networks is their adaptive nature, where “learning by example” replaces conventional “programming by different cases” in solving problems.

There are three major learning paradigms, each corresponding to a particular abstract learning task. These are supervised learning, unsupervised learning and reinforcement learning.

Most of the algorithms used in training artificial neural networks employ some form of gradient descent. This is done by simply taking the derivative of the cost function with respect to the network parameters and then changing those parameters in a gradient-related direction. Evolutionary methods, gene expression programming, simulated annealing, expectation-maximization, non-parametric methods and particle swarm optimization are some commonly used methods for training neural networks.

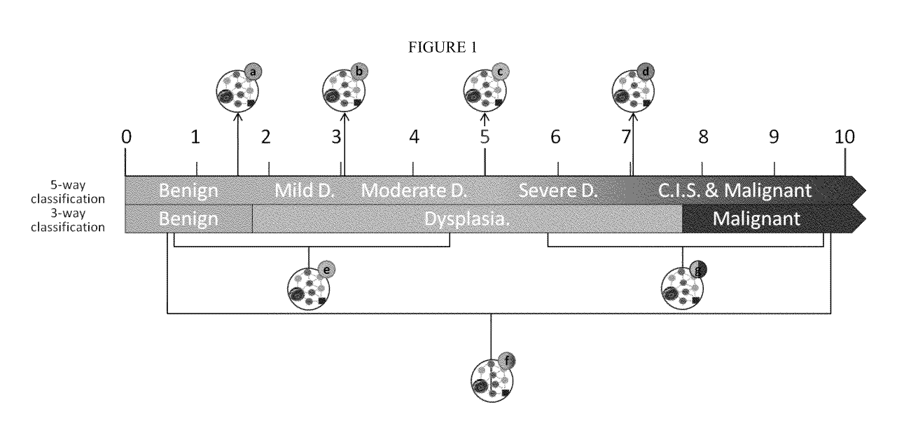

As an example, in FIG. 1, “nodes” a-d correspond to nodes created from 5-way ordinal binary classification—essentially a yes or no answer to a question, such as is the cell round? Node (a) discriminates between benign and all other categories above (mild dysplasia (D), moderate D, severe D, OSCC and CIS); (b) discriminates between (benign and mild D) vs. (mod D, severe D, OSCC and CIS); (c) discriminates between (benign, mild D and mod D) vs. (severe D, OSCC and CIS); (d) discriminates between (benign, mild D, mod D, severe D) and (OSCC and CIS).

Nodes e-g correspond to nodes created from 3-way ordinal binary classification. (e) discriminates between the benign category and the dysplastic category (including mild D, mod D, and severe D); (f) discriminates between the benign category versus (OSCC and CIS); and (g) discriminates between the dysplastic category (including mild D, mod D, and severe D) and (OSCC and CIS).

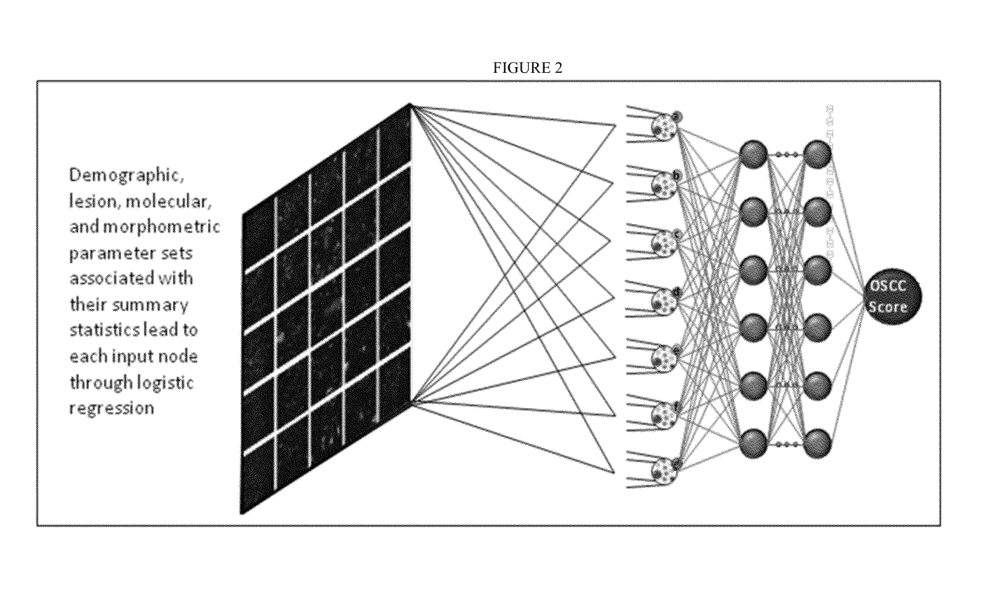

Nodes can include demographic and smoking/alcohol information or can be combined to other nodes containing this information as input. All nodes are combined as is exemplified with the artificial neural network (ANN) architecture, which is one of the possible algorithm to be used here, shown in FIG. 2.

The ANN consists of all the nodes as input in the input layer. The blue nodes in the center correspond to the hidden layers performing radial basis activation functions. In a feed forward neural network the number of hidden layers and nodes can be varied to maximize the fitness during training Finally the output layer consists of a single score which will be normalized to be between 1 and 10. Of course, any range could be used but 1-10 is fairly typical.

This disclosed method can be used by clinicians as the result of lesion analysis will come to them without the input of a pathologist for their interpretation in a single score that will be associated with clear clinical decision rules. For example, score higher than 5 means patient needs to be referred to scalpel biopsy. Or, a score between 3 and 5 means patient needs to be seen in one month for repeat brush biopsy. These clinical decision rules have not been definitively established yet, but a clear quantitative score such as one produced here will empower clinicians to make these decisions with more assurance.

None of the adjunctive techniques currently used for screening of oral lesions are quantitative. This oral cancer scoring system is the first with sufficient power to do so.

A clinical trial has been run with recruitment completed. Analysis is ongoing, but points to clear high performance combination of morphological, molecular, demographic and behavioral parameters to define the nodes presented in this disclosure.

Multiple methods will be employed and compared based on machine learning, including but not limited to multivariate analysis, ANN, regression tree, etc. and the model will be built and tested with ⅔ of the data as training, and ⅓ kept blind for validation. Other machine based learning analysis include decision tree learning, association rule learning, artificial neural networks, genetic programming, inductive logic programming, support vector machines, clustering, Bayesian networks, reinforcement learning, representation learning, similarity and metric learning, and sparse dictionary learning.

One limitation is currently related to the use of the output score and the creation of associated clinical decision rules, as the oral community is in a transition to explore alternative classification to the WHO guidelines. However, the algorithm is being built with nodes from all possible classifications, and therefore will be relevant to both models.

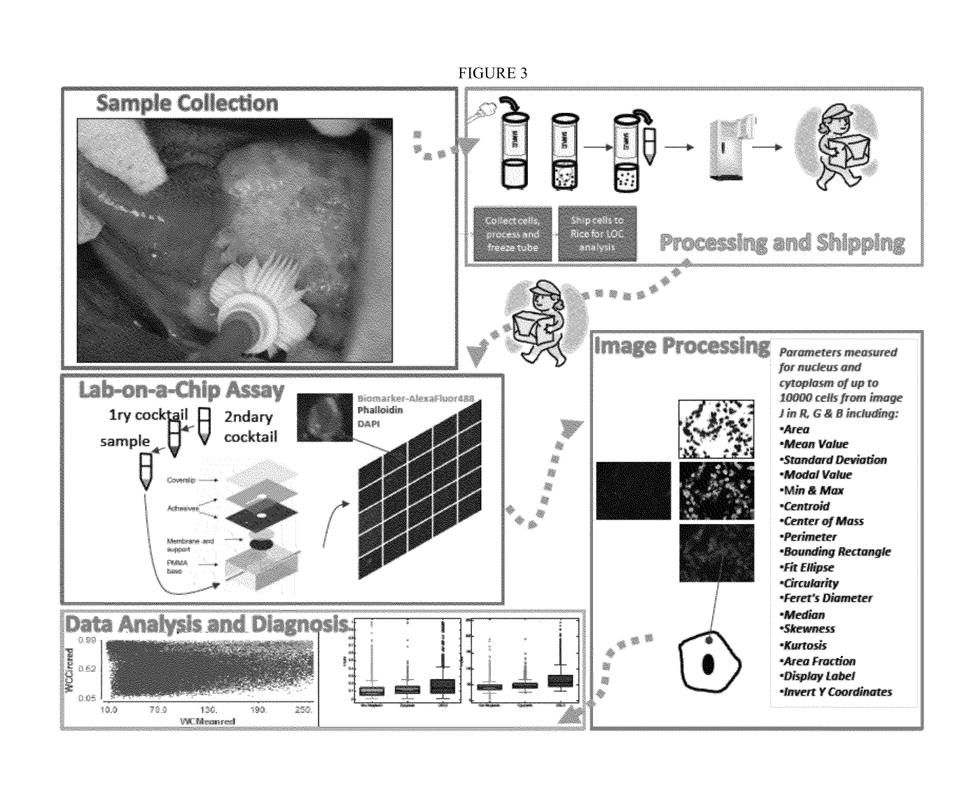

Methods and exemplary data are provided in FIG. 3-13. Codes were assigned to the different classes as Normal (1), Benign (2), Mild Dysplasia (3), Moderate Dysplasia (4), Severe Dysplasia (5), Carcinoma in Situ (6), and Malignant (7). As such, the report will focus here on discriminations according to the ordinal 3-way classification in Benign v. Malignant (CLASS BvM); Dysplastic v. Malignant (CLASS DvM); Benign v. Dysplastic (CLASS BvD).

Also featured will be discriminations according to the 5-way classification (Normals not considered here, and CIS part of malignant) in CLASS x23 (non cases<=2 i.e. Benign, and cases>=3 including Mild Dysplasia, Moderate Dysplasia, Severe Dysplasia, CIS, and Malignant); CLASS x34 (non cases<=3 including Benign and Mild Dysplasia, and cases>=4 including Moderate Dysplasia, Severe Dysplasia, CIS, and Malignant), and CLASS x45 where the case/non case threshold is between Moderate and Severe Dysplasias.

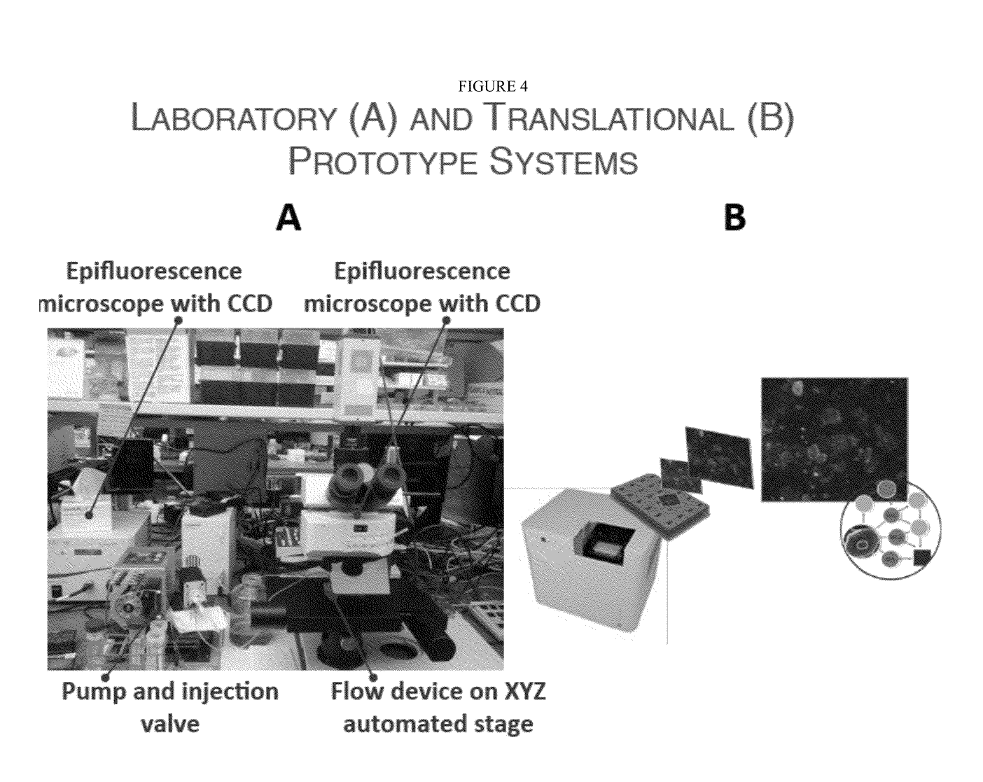

FIG. 3 shows the overall process, from sample collection to lab-on-chip analysis, to image collection and analysis. FIG. 4 shows exemplary prototype and commercial equipment used in the lab-on-chip assays, but is described in more detail in other applications by McDevitt.

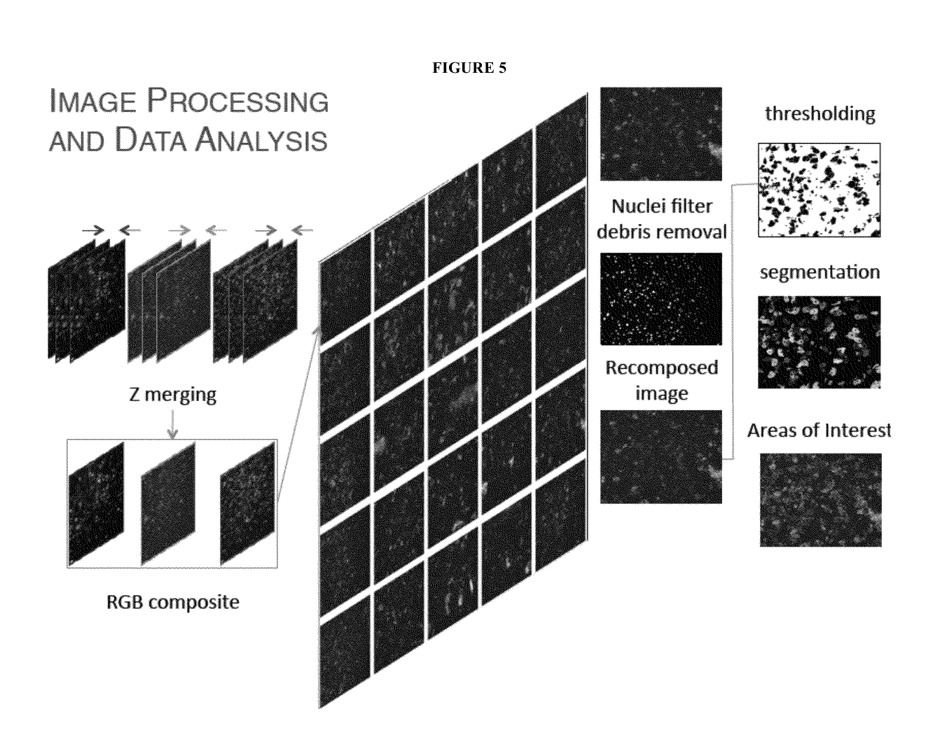

FIG. 5 shows the image analysis process used to collect the morphometric and biomarker data. It is described more completely in WO2012065117, but FIG. 5. basically shows a conceptual flowchart of the image collection and conditioning process before measurements are performed on individual cell profiles. A series of images spanning the desired field and across several focal planes are collected and merged into single in-focus image sequence. Image filtering (such as background subtraction and debris removal) can be performed before final thresholding. Individual cellular outlines are profiled by thresholding and segmenting each image based on pre-established cutoff values for cytoplasmic and nuclear staining intensity. These profiles are compiled into a set of regions of interest (ROIs) which are then used to extract biomarker and morphometric parameters for each cell.

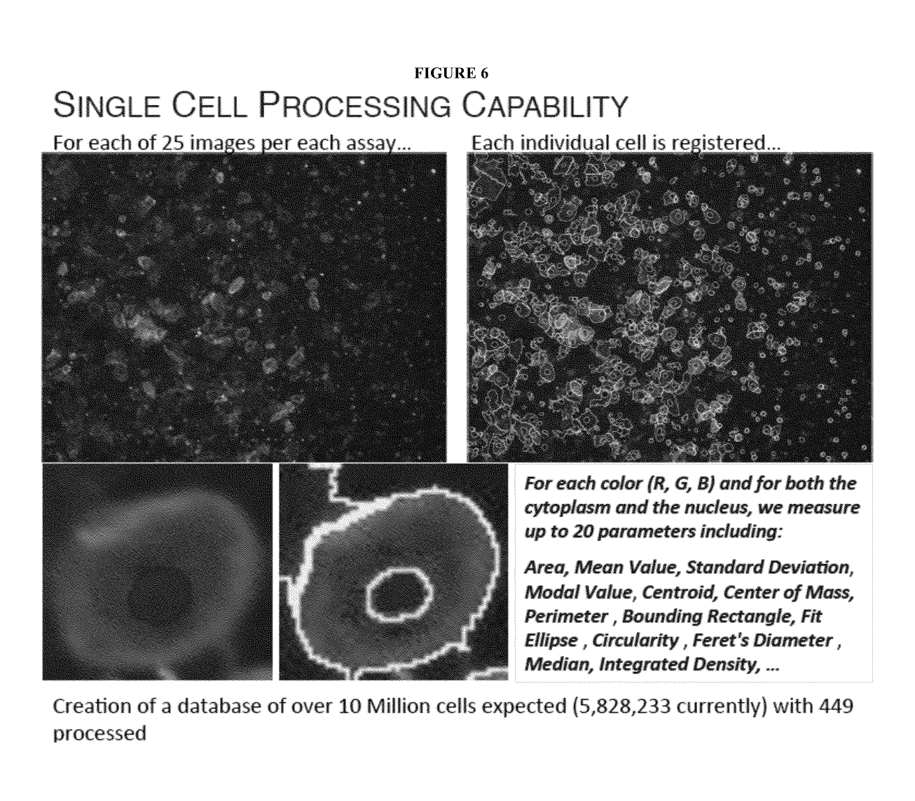

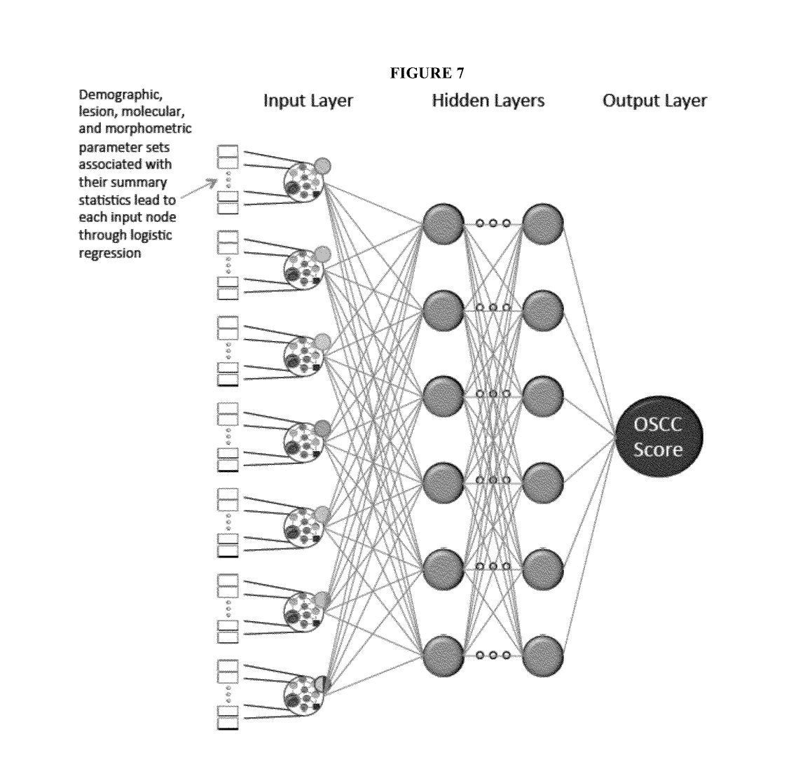

FIG. 6 shows exemplary image capture, wherein many cells from each photograph are outlined, and various types of data captured on a per cell basis. FIG. 7 shows a visual depiction of the back-end data processing components of the neural net architecture used to generate the OSCC risk assessment score. Input nodes are passed to an input layer based on logistic regression, the number of hidden layers and computing nodes are optimized, and a normalized risk assessment score between 1 and 10 is outputted.

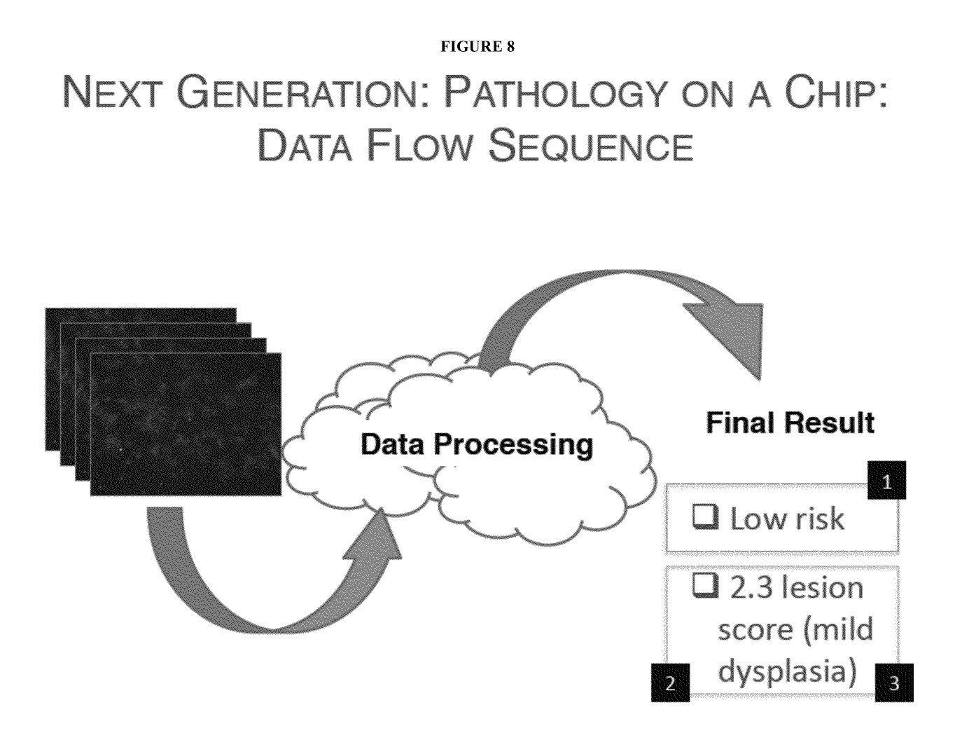

FIG. 8 shows the next generation system wherein images are collected at one site, analyzed in the cloud, and the final result sent to the pathologist or physician. This system is not yet in place, but is expected to be implanted when the clinical trial data analysis is completed and will be commercially available in 2015 or 2016.

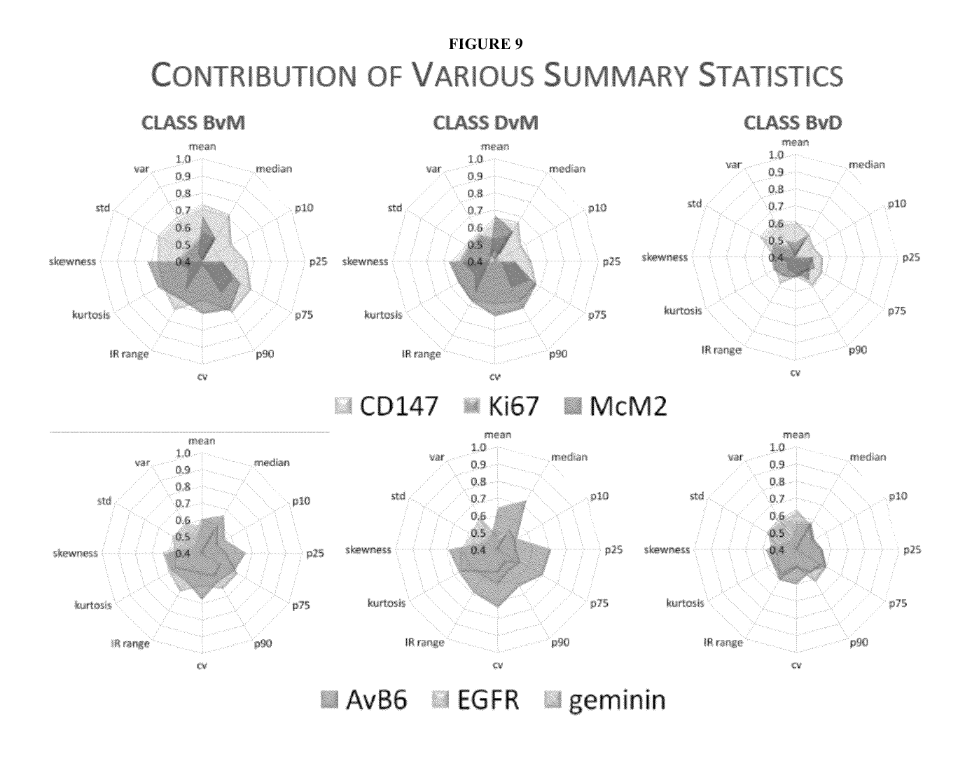

FIG. 9 shows a radar chart that illustrates the contributions of various summary statistics (mean, median, standard deviation, variance, etc.) of the cellular populations for different biomarkers corresponding to three different model classes: benign vs malignant (BvM), dysplastic vs malignant (DvM), and benign vs dysplastic (BvD). The values on the radar chart are computed area's under ROC curves (AUC's) for each model parameter. Diagnostic perfection in this case is represented with an area under the curve of 1.0, that is the outer extreme of the web. Poor performance would be closer to the random value, which is located at the center of the web. Since an entire cellular population is used to evaluate a patient's risk assessment, it is better to look at various population statistics that have potential to provide more granularity, rather than relying solely on an arithmetic mean value for each biomarker or morphological parameter. This data reveals that various biomarker parameters as extracted from the images can yield strong capabilities to help diagnose the status of oral cancer disease progression.

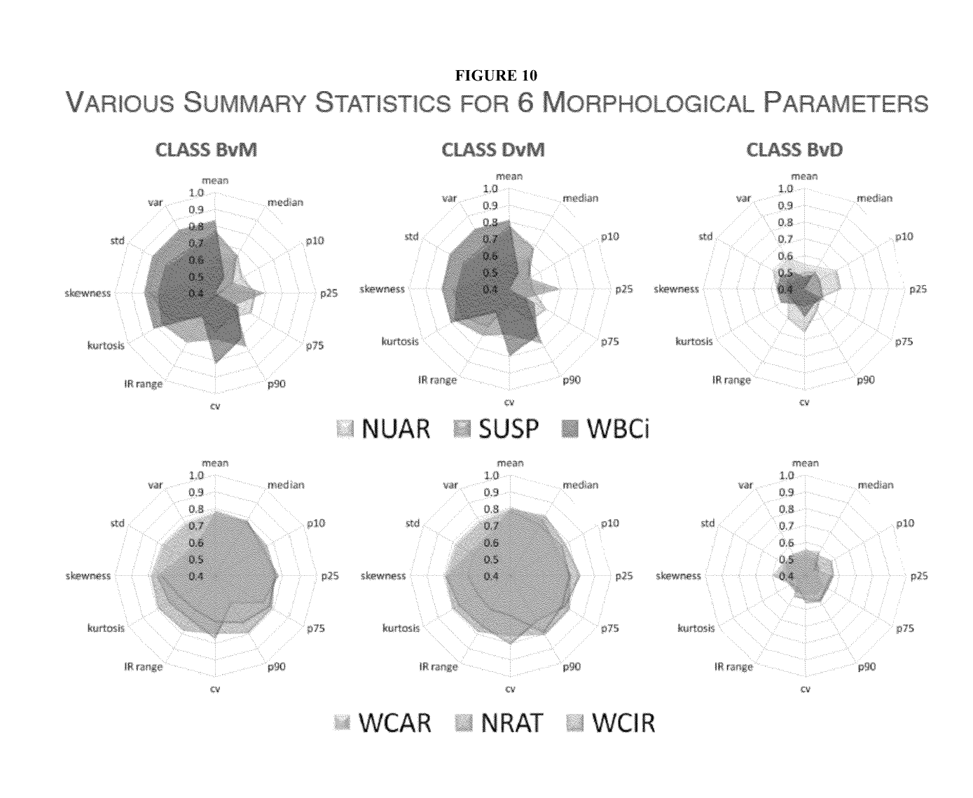

FIG. 10 shows a similar radar chart as seen in FIG. 9, but illustrates the contributions of various summary statistics of the cellular populations for 6 morphological parameters corresponding to the same three model classes.

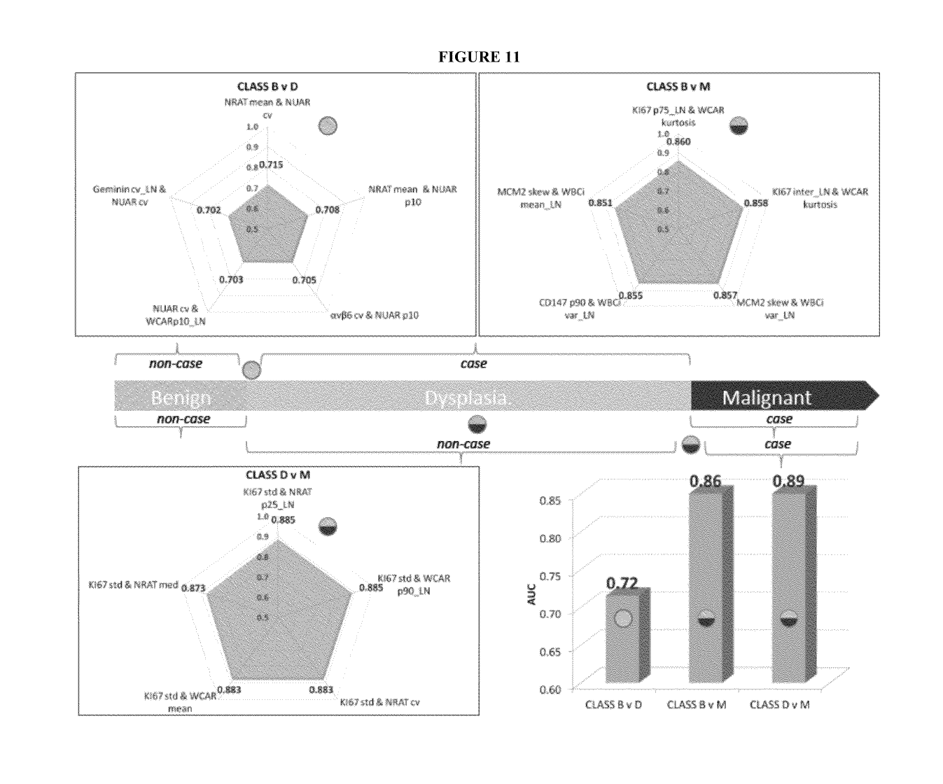

FIG. 11 shows the relative AUC values for the 5 best 2-parameter models for 3 model classes: BvD, BvM, and DvM. Additionally, a bar graph is included to emphasize the best AUC value from the given 2-parameter models with regard to the different model classes.

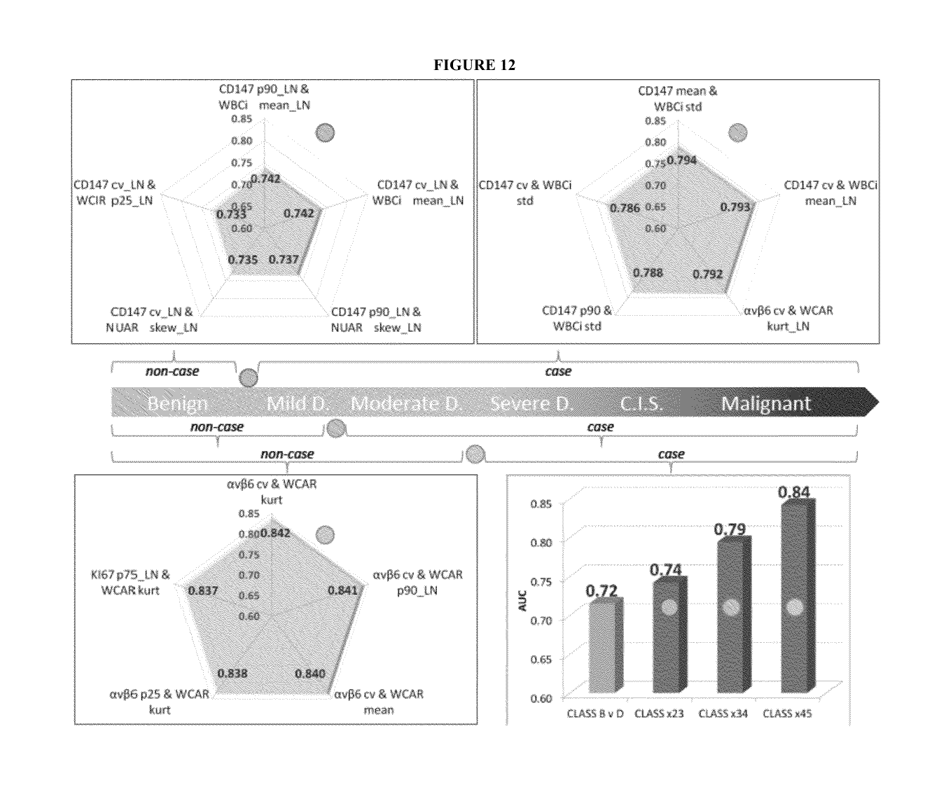

FIG. 12 shows the relative AUC values for the 5 best 2-parameter models for 3 model classes: x23, x34, and x45. Additionally, a bar graph is included to emphasize the best AUC value from the given 2-parameter models with regard to the different model classes.

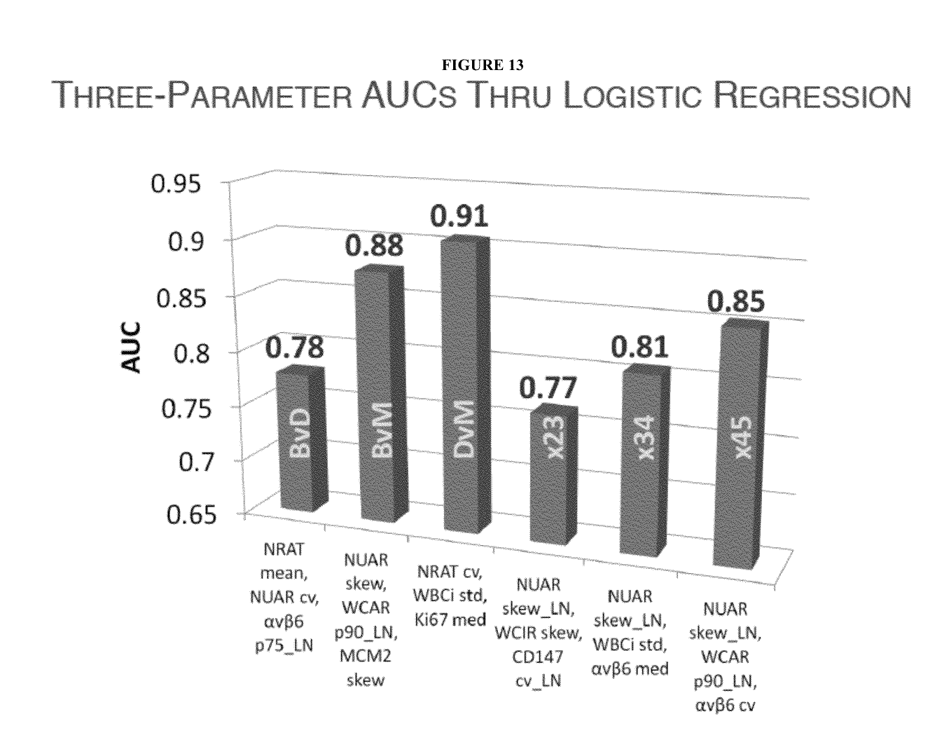

FIG. 13 shows a bar graph that displays the AUC's for ROC curves derived from 6 different 3-parameter models. The AUC's provide a general measure of the goodness of model fit, and are useful in evaluating the performance of competing models in a model selection process. For example, in FIG. 13, the model incorporating the coefficient of variation of the nuclear-to-cytoplasmic ratio, standard deviation of infiltrated white blood cells, and the median of nuclear biomarker Ki67 has a combined AUC of 0.91 when differentiating between dysplastic and malignant cases.

Our results to date show that several inputs are particularly relevant to classifying a disease state including MCM2, AVB6, cell area, nuclear area, and nuclear-to-cytoplasm ratio. Additional inputs that were valuable in disease classification include biomarkers EGFR, CD147 and KI67 and morphometric parameters relating to cell shape and/or roundness. Our data to date shows the best models produce 88-90% sensitivity and 63-70% specificity, although these data analysis is ongoing.

The following references are incorporated by reference in their entireties for all purposes:

US8257967, WO03090605, US20060073585, US2006079000, US2006234209, WO2004009840, WO2004072097, US7781226, US8101431, US8105849, US2006257854, US20060257941, US2006257991, WO2005083423, WO2005085796, WO2005085854, WO2005085855, WO2005090983, US8377398, WO2007053186, US2010291431, WO2007002480, US2008050830, WO2007134191, US2008038738, WO2007134189, US2008176253, US2008300798, WO2008131039, US2012208715, WO2011022628, US2013130933, WO2012021714, US2013295580, WO2012065117, US2013274136, WO2012065025, WO2012154306, US2012322682.

1) A method of scoring oral cancer lesions comprising:
a) inputting the following data points into a computer:
i) one or more morphological characteristics from individual oral cells from a patient, said morphological characteristics selected from nuclear area, cell area, cell circularity, cell aspect ratio, and cell roundness;
ii) one or more of gender, age, alcohol intake, and smoking status of said patient;
iii) one or more biomarker levels from individual oral cells from said patient, said biomarker selected from the group consisting of alpha V beta 6 (AVB6), Epidermal Growth Factor Receptor (EGFR), Ki67, Geminin, Mini Chromosome Maintenance protein (MCM2), beta catenin, EMPPRIN, CD147;
b) calculating a risk score based on each of the above inputs, said risk score allowing a user to distinguish at least the following: i) benign lesions, ii) dysplastic lesions, and iii) cancerous lesions; and
c) displaying said risk score on an output device.

2) The method of claim 1, wherein said calculation results in 4-way, 5-way or 6-way ordinal scale of disease progression.

3) The method of claim 1, said calculation allowing a user to distinguish the following: 1) benign lesions, 2) mild dysplasia, 3) moderate dysplasia, 4) severe dysplasia, and 5) oral squamous cell carcinoma (OSCC).

4) The method of claim 1, said calculation allowing a user to distinguish the following: 1) benign lesions, 2) mild dysplasia, 3) moderate dysplasia, 4) severe dysplasia, and 5) oral squamous cell carcinoma (OSCC) combined with carcinoma in situ (CIS).

5) The method of claim 1, said calculation based on artificial neural nets, logistic regression, linear discriminate analysis, or random forests.

6) The method of claim 1, said calculation based on feedforward artificial neural nets.

7) The method of claim 1, said calculation based on prior artificial neural network model training using data points from patients with known disease states.

8) The method of claim 1, said calculation based on continued neural network model training using data points from patients with known disease states and outcomes.

9) The method of claim 1, wherein each inputted data point from i), ii) and ii) each correspond to a node, and each node is linked to serve as input in a neural network in creating a single output risk score on a continuous scale between 1 and 10.

10) The method of claim 1, wherein said calculation is based on inputting nodes into an input layer, said nodes obtained through logistic regression of all possible classifications of patient samples having known disease states according to at least 3-way classifications; optimizing the artificial neural network as to the number of hidden layers and computing nodes, and outputting a normalized score between 1 and 10, 1 corresponding to benign and 10 corresponding to malignant.

11) The method of claim 1, said calculation including:
Oral Cancer Risk Score=a0+a1×P1+a2×P2+ . . . an X Pn, where each of P1, P2, . . . Pn is a node of a logistic regression model, where n is the number of nodes and where a0-an is a weight factor determined by training with input data from patients having known disease status.

12) A method of scoring oral cancer lesions comprising:
a) inputting the following data points into a computer:
i) two, three or more morphological characteristics from individual oral cells from a patient, said morphological characteristics selected from cell area, nuclear area, cell circularity, cell aspect ratio, and cell roundness;
ii) two, three or more of gender, age, alcohol intake, and smoking status of said patient;
iii) two, three or more biomarker levels from individual oral cells from said patient, said biomarker selected from the group consisting of AVB6, EGFR, Ki67, MCM2, beta catenin, EMPPRIN, and CD147; and
b) calculating a risk score based on each of the above inputs, wherein said calculation is based on logistic regression or neural network training using data points from patients with known disease status, said risk score providing at least 3 disease classifications; and
c) displaying said risk score on an output device.

13) The method of claim 12, said risk score allowing a user to distinguish the following: 1) benign conditions, 2) dysplastic conditions, 3) moderate disease, and 4) high risk disease.

14) The method of claim 12, said calculation including levels of AVB6 and MCM2.

15) The method of claim 12, said calculation including cell area, nuclear area, and levels of AVB6, MCM2, Ki67 and CD147.

16) A method of detecting oral cancer and scoring oral lesions comprising:
a) obtaining an oral sample from a patient suspected of having an oral lesion, said oral sample containing a plurality of cells;
b) determining a cell area, a nuclear area, and a level of AVB6, MCM2, Ki67 and CD147 in each of said plurality of cells;
c) inputting the following data points into a computer:
i) said determined cell area and said determined nuclear area for each of said plurality of cells;
ii) three or more of gender, age, alcohol intake, and smoking status of said patient;
iii) said determined AVB6, MCM2, Ki67 and CD 147 levels for each of said plurality of cells; and
d) calculating a risk score based on each of the above data points; and
e) displaying said risk score on an output device, wherein said risk score distinguishes at least three disease states.

17) The method of claim 16, said calculation allowing a user to distinguish between benign conditions, mild dysplastic conditions, moderate dysplastic conditions, severe dysplastic conditions and cancerous conditions.

18) The method of claim 16, said calculation including cell area, nuclear area, and levels of AVB6 and MCM2.

19) The method of claim 16, said calculation including cell area, nuclear area, and levels of AVB6, MCM2, Ki67 and CD147.

20) The method of claim 16, said calculation using neural net data analysis techniques.
